(19) United States
(12) Patent Application Publication
(10) Pub. No.: US 2022/0346710 A1
(43) Pub. Date: Nov. 3, 2022

(54) LEARNED MODEL GENERATING METHOD, PROCESSING DEVICE, AND STORAGE MEDIUM

(71) Applicant: GE Precision Healthcare LLC, Wauwatosa, WI (US)

(72) Inventors: Shotaro Fuchibe, Tokyo (JP); Yotaro Ishihara, Tokyo (JP)

(21) Appl. No.: 17/731,368

(22) Filed: Apr. 28, 2022

(30) Foreign Application Priority Data
Apr. 28, 2021 (JP) 2021-076887

(51) Int. Cl. A61B 5/00; A61B 5/11; G06V 40/10; G06V 10/82; G06T 7/70; G06N 3/08

(52) U.S. Cl. CPC A61B 5/4872; A61B 5/1116; A61B 5/1128; G06V 40/10; G06V 10/82; G06T 7/70; G06N 3/08; G06T 2207/10081; G06T 2207/20081; G06T 2207/20084; G06T 2207/30196

(57) ABSTRACT

To provide a technology that can easily acquire body weight information of a patient. A processing part that deduces the body weight of a patient based on a camera image of the patient lying on a table of a CT device, including a generating part that generates an input image based on the camera image, and a deducing part that deduces the body weight of the patient when the input image is input into a learned model. The learned model is generated by a neural network executing learning using a plurality of learning images C1 to Cn generated based on a plurality of camera images, and a plurality of correct answer data G1 to Gn corresponding to the plurality of learning images C1 to Cn, where each of the plurality of correct answer data G1 to Gn represents a body weight of a human included in a corresponding learning image.

### CROSS-REFERENCE TO RELATED APPLICATIONS

This application claims priority to Japanese Patent Application No. 2021-076887, filed on Apr. 28, 2021, the disclosure of which is incorporated herein by reference in its entirety.

### BACKGROUND

The present invention relates to a method of generating a learned model for deducing body weight, a processing device that executes a process for determining body weight of an imaging subject lying on a table, and a storage medium storing a command for causing a processor to execute the process for determining body weight.

An x-ray computed tomography (CT) device is known as a medical device that non-invasively captures images of the inside of a patient. X-ray CT devices can capture images of a site to be imaged in a short period of time, and therefore have become widespread in hospitals and other medical facilities.

On the other hand, CT devices use X-rays to examine patients, and as CT devices become more widespread, there is increasing concern about patient exposure during examinations. Therefore, it is important to control patient exposure dose from the perspective of reducing the patient exposure dose from X-rays as much as possible and the like. Therefore, technologies to control the dose have been developed. For example, Patent Document 1 discloses a dose control system.

In recent years, dose control has become stricter based on guidelines by the Ministry of Health, Labour and Welfare, and these guidelines state that dose control should be based on the diagnostic reference level (DRL). The dose must be controlled with reference to the guidelines for diagnostic reference levels. Furthermore, different patients have different physiques, and therefore, it is important to control not only the exposure dose to which the patient is subjected during a CT scan but also the patient body weight information in order to control the dose for each patient. Therefore, medical institutions obtain body weight information of each patient and record the information in the RIS (Radiology Information System).

In medical institutions, for example, the body weight of a patient is measured by a weight scale before a CT scan, in order to obtain patient body weight information. Once the body weight of the patient is measured, the measured body weight is recorded in the RIS. However, it is not always possible to measure the body weight of the patient on a weight scale for every CT scan. Therefore, the body weight information recorded in the RIS may be out of date, and it is not desirable to control the dose with the outdated body weight information. Furthermore, there is also a problem where if the patient is using a wheelchair or stretcher, body weight measurement itself is not easy.

Therefore, there is demand for a technology that can easily acquire body weight information of a patient.

### SUMMARY

A first aspect of the present invention is a learned model generating method of generating a learned model that outputs a body weight of an imaging subject when an input image of the imaging subject lying on a table of a medical device is input, where a neural network generates the learned model by executing learning using: a plurality of learning images generated based on a plurality of camera images of a human lying on a table of a medical device, and a plurality of correct answer data corresponding to the plurality of learning images, where each of the plurality of correct answer data represents a body weight of a human included in a corresponding learning image.

A second aspect of the present invention is a processing device that executes a process of determining a body weight of an imaging subject based on a camera image of the imaging subject lying on a table of a medical device.

A third aspect of the present invention is a storage medium, including one or more non-volatile, computer-readable storage media storing one or more commands that can be executed by one or more processors, where the one or more commands causes the one of more processors to execute a process of determining a body weight of an imaging subject based on a camera image of the imaging subject lying on a table of a medical device.

A fourth aspect of the present invention is a medical device that executes a process of determining a body weight of an imaging subject based on a camera image of the imaging subject lying on a table of a medical device.

A fifth aspect of the present invention is a learned model that outputs a body weight of an imaging subject when an input image of the imaging subject lying on a table of a medical device is input, where the learned model is generated by a neural network executing learning using: a plurality of learning images generated based on a plurality of camera images of a human lying on a table of a medical device, and a plurality of correct answer data corresponding to the plurality of learning images, where each of the plurality of correct answer data represents a body weight of a human included in a corresponding learning image.

A sixth aspect of the present invention is a learned model generating device that generates a learned model that outputs a body weight of an imaging subject when an input image of the imaging subject lying on a table of a medical device is input, where a neural network generates the learned model by executing learning using: a plurality of learning images generated based on a plurality of camera images of a human lying on a table of a medical device, and a plurality of correct answer data corresponding to the plurality of learning images, where each of the plurality of correct answer data represents a body weight of a human included in a corresponding learning image.

There is a certain correlation between human physique and body weight. Therefore, a learning image can be generated based on a camera image of a human, and the learning image can be labeled with the body weight of a human as correct answer data. Then, a neural network can execute learning using the learning image and correct answer data to generate a learned model that can deduce body weight. Furthermore, medical devices include medical devices that perform scanning with a patient lying on a table, such as CT devices, MM devices, and the like. Therefore, if a camera for acquiring a camera image of the patient lying on the table is prepared, a camera image including the patient can be acquired. Thus, based on the acquired camera image, an input image to input to the learned model can be generated, and the input image can be input to the learned model to deduce the body weight of the patient.

Therefore, the body weight of the patient can be deduced without having to measure the body weight of the patient for each examination, and thus the body weight of the patient at the time of the examination can be managed.

Furthermore, if the BMI and height are known, the body weight can be calculated. Therefore, body weight information can also be obtained by deducing height instead of body weight, and calculating the body weight based on the deduced height and BMI.

### BRIEF DESCRIPTION OF DRAWINGS

FIG. 1 is an explanatory diagram of a hospital network system. An explanatory diagram of a hospital network system.

FIG. 2 is a schematic view of an X-ray CT device.

FIG. 3 is an explanatory diagram of a gantry 2, a table 4, and an operation console 8.

FIG. 4 is a diagram showing main functional blocks of a processing part 84.

FIG. 5 is a diagram showing a flowchart of a learning phase.

FIG. 6 is an explanatory diagram of a learning phase.

FIG. 7 is a diagram showing an examination flow.

FIG. 8 is a diagram illustrating a schematic view of a generated input image 61.

FIG. 9 is an explanatory diagram of a deducing phase.

FIG. 10 is a diagram illustrating an input image 611.

FIG. 11 is an explanatory diagram of a method of confirming to an operator whether or not a body weight is updated.

FIG. 12 is an explanatory diagram of an example of various data transmitted to a PACS 11.

FIG. 13 is a diagram showing main functional blocks of the processing part 84 according to embodiment 2.

FIG. 14 is a diagram schematically illustrating learning images CI1 to CIn.

FIG. 15 is a diagram showing an examination flow according to embodiment 2.

FIG. 16 is a diagram schematically illustrating an input image 62.

FIG. 17 is an explanatory diagram of a deducing phase of deducing the height of a patient 40.

FIG. 18 is an explanatory diagram of a method of confirming whether or not a body weight and height are updated.

FIG. 19 is an explanatory diagram of learning images and correct answer data prepared for postures (1) to (4).

FIG. 20 is an explanatory diagram of step ST2.

FIG. 21 is a diagram schematically illustrating an input image 64.

FIG. 22 is an explanatory diagram of a deducing phase of deducing the body weight of the patient 40.

FIG. 23 is a diagram showing main functional blocks of the processing part 84 according to embodiment 4.

FIG. 24 is an explanatory diagram of step ST2.

FIG. 25 is a diagram showing an examination flow of the patient 40 according to embodiment 4.

FIG. 26 is an explanatory diagram of a deducing phase of deducing body weight.

### DETAILED DESCRIPTION

Embodiments for carrying out the invention will be described below, but the present invention is not limited to the following embodiments.

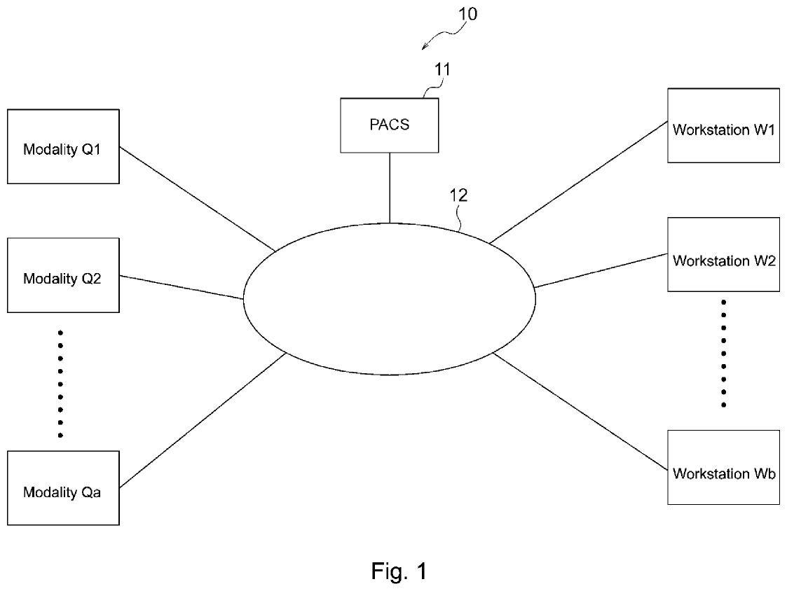

FIG. 1 is an explanatory diagram of a hospital network system. A network system 10 includes a plurality of modalities Q1 to Qa. Each of the plurality of modalities Q1 to Qa is a modality that performs patient diagnosis, treatment, and the like.

Each modality is a medical system with a medical device and an operation console. The medical device is a device that collects data from a patient, and the operation console is connected to the medical device and is used to operate the medical device. The medical device is a device that collects data from a patient. Examples of medical devices that can be used include simple X-ray devices, X-ray CT devices, PET-CT devices, MRI devices, MM-PET devices, mammography devices, and various other devices. Note that in FIG. 1, the system 10 includes a plurality of modalities, but may include a single modality instead of a plurality of modalities.

Furthermore, the system 10 also has PACS (Picture Archiving and Communication Systems) 11. The PACS 11 receives an image and other data obtained by each modality via a communication network 12 and stores the received data. Furthermore, the PACS 11 also transfers the stored data via the communication network 12 as necessary.

Furthermore, the system 10 has a plurality of workstations W1 to Wb. The workstations W1 to Wb include, for example, workstations used in hospital information systems (HIS), radiology information systems (RIS), clinical information systems (CIS), cardiovascular information systems (CVIS), library information systems (LIS), electronic medical record (EMR) systems, and/or other image and information management systems and the like, and workstations used for image inspection work by an image interpreter.

The network system 10 is configured as described above. Next, an example of a configuration of the X-ray CT device, which is an example of a modality, will be described.

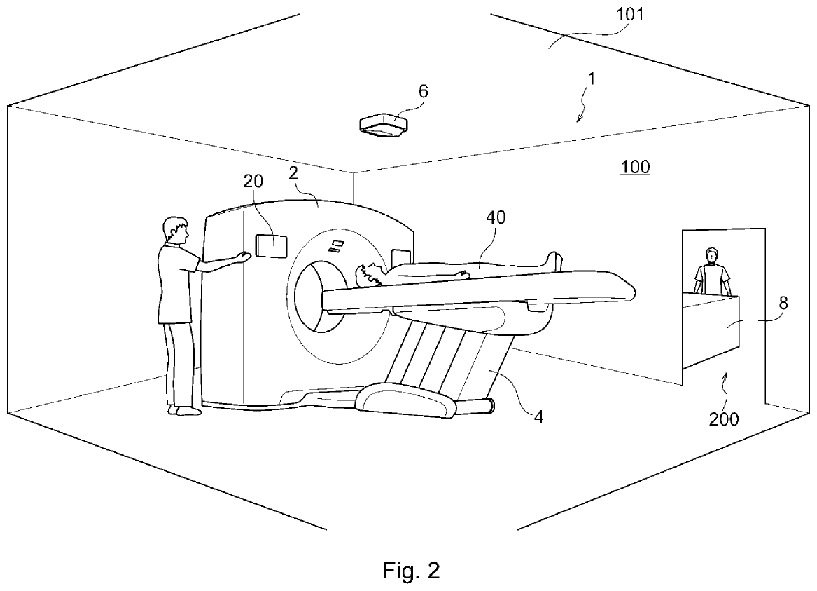

FIG. 2 is a schematic view of the X-ray CT device. As illustrated in FIG. 2, an X-ray CT device 1 includes a gantry 2, a table 4, a camera 6, and an operation console 8.

The gantry 2 and table 4 are installed in a scan room 100. The gantry 2 has a display panel 20. An operator can input an operation signal to operate the gantry 2 and table 4 from the display panel 20. The camera 6 is installed on a ceiling 101 of the scan room 100. The operation console 8 is installed in an operation room 200.

A field of view of the camera 6 is set to include the table 4 and a perimeter thereof. Therefore, when the patient 40, who is an imaging subject, lies on the table 4, the camera 6 can acquire a camera image including the patient 40.

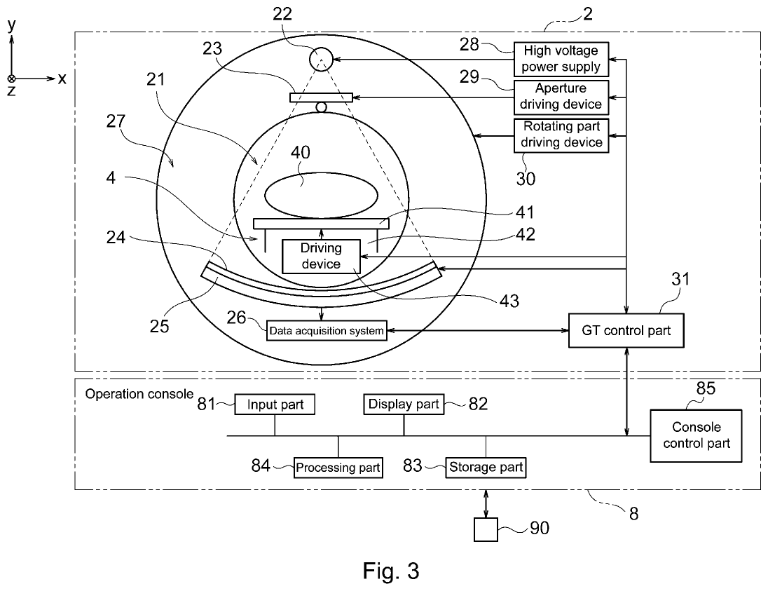

Next, the gantry 2, table 4, and operation console 8 will be described with reference to FIG. 3.

FIG. 3 is an explanatory diagram of the gantry 2, the table 4, and the operation console 8. The gantry 2 has an inner wall that demarcates a bore 21, which is a space in which the patient 40 can move.

Furthermore, the gantry 2 has an X-ray tube 22, an aperture 23, a collimator 24, an X-ray detector 25, a data acquisition system 26, a rotating part 27, a high-voltage power supply 28, an aperture driving device 29, a rotating part driving device 30, a GT (Gantry Table) control part 31, and the like.

The X-ray tube 22, aperture 23, collimator 24, X-ray detector 25, and data acquisition system 26 are mounted on the rotating part 27.

The X-ray tube 22 irradiates the patient 40 with X-rays. The X-ray detector 25 detects the X-rays emitted from the X-ray tube 22. The X-ray detector 25 is provided on an opposite side of the X-ray tube 22 from the bore 21.

The aperture 23 is disposed between the X-ray tube 22 and the bore 21. The aperture 23 shapes the X-rays emitted from an X-ray focal point of the X-ray tube 22 toward the X-ray detector 25 into a fan beam or a cone beam.

The X-ray detector 25 detects the X-rays transmitted through the patient 40. The collimator 24 is disposed on the X-ray incident side to the X-ray detector 25 and removes scattered X-rays.

The high voltage power supply 28 supplies high voltage and current to the X-ray tube 22. The aperture driving device 29 drives the aperture 23 to deform an opening thereof. The rotating part driving device 30 rotates and drives the rotating part 27.

The table 4 has a cradle 41, a cradle support 42, and a driving device 43. The cradle 41 supports the patient 40, who is an imaging subject. The cradle support 42 movably supports the cradle 41 in the y direction and z direction. The driving device 43 drives the cradle 41 and cradle support 42. Note that herein, a longitudinal direction of the cradle 41 is a z direction, a height direction of the table 4 is a y direction, and a horizontal direction orthogonal to the z direction and y direction is an x direction.

A GT control part 31 controls each device and each part in the gantry 2, the driving device 43 of the table 4, and the like.

The operation console 8 has an input part 81, a display part 82, a storage part 83, a processing part 84, a console control part 85, and the like.

The input part 81 includes a keyboard, a pointing device, and the like for accepting instructions and information input from an operator and performing various operations. The display part 82 displays a setting screen for setting scan conditions, camera images, CT images, and the like and is, for example, an LCD (Liquid Crystal Display), OLED (Electro-Luminescence) display, or the like.

The storage part 83 stores a program for executing various processes by a processor. Furthermore, the storage part 83 also stores various data, various files, and the like. The storage part 83 has a hard disk drive (HDD), solid state drive (SSD), dynamic random access memory (DRAM), read only memory (ROM), and the like. Furthermore, the storage part 83 may also include a portable storage medium 90 such as a CD (Compact Disk), DVD (Digital Versatile Disk), or the like.

The processing part 84 performs an image reconfiguring process and various other operations based on data of the patient 40 acquired by the gantry 2. The processing part 84 has one or more processors, and the one or more processors execute various processes described in the program stored in the storage part 83.

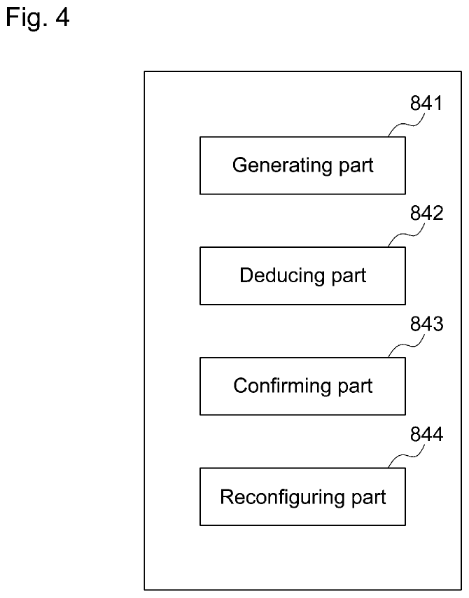

FIG. 4 is a diagram showing main functional blocks of the processing part 84. The processing part 84 has a generating part 841, a deducing part 842, a confirming part 843, and a reconfiguring part 844.

The generating part 841 generates an input image to be input to the learned model based on a camera image. The deducing part 842 inputs the input image to the learned model to deduce the body weight of the patient. The confirming part 843 confirms to the operator whether or not to update the deduced body weight. The reconfiguring part 844 reconfigures a CT image based on projection data obtained from a scan.

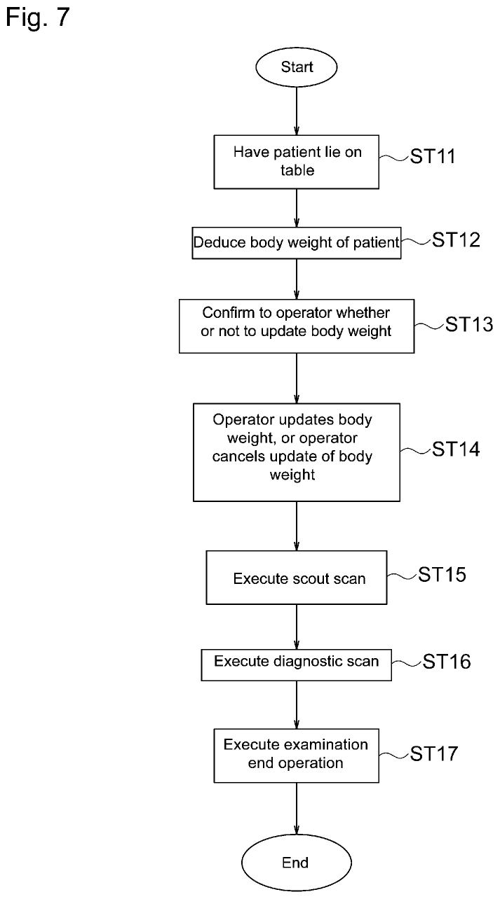

Note that details of the generating part 841, deducing part 842, confirming part 843, and reconfiguring part 844 will be described in each step of an examination flow (refer to FIG. 7) described later.

A program for executing the aforementioned functions is stored in the storage part 83. The processing part 84 implements the aforementioned functions by executing the program. One or more commands that can be executed by one or more processors are stored in the storage part 83. The one or more commands cause one or more processors to perform the following operations (a1) to (a4): (a1) Generating an input image to be input to the learned model based on a camera image (generating part 841), (a2) Inputting the input image to the learned model to deduce the body weight of the patient (deducing part 842), (a3) Confirming to the operator whether or not to update the body weight (confirming part 843), (a4) Reconfiguring a CT image based on projection data (reconfiguring part 844).

The processing part 84 of the console 8 can read the program stored in the storage part 83 and execute the aforementioned operations (a1) to (a4).

The console control part 85 controls the display part 82 and the processing part 84 based on an input from the input part 81.

The X-ray CT device 1 is configured as described above.

FIG. 3 illustrates a CT device as an example of a modality, but hospitals are also equipped with medical devices other than CT devices, such as Mill devices, PET devices, and the like.

In recent years, there has been a demand for strict control of patient exposure dose when performing examinations that use X-rays, such as CT scans and the like. In medical institutions, for example, the body weight of a patient is measured by a weight scale before a CT scan, in order to obtain patient body weight information. Once the body weight of the patient is measured, the measured body weight is recorded in the RIS. However, it is not always possible to measure the body weight of the patient on a weight scale for every CT scan. Therefore, the body weight information recorded in the RIS may be out of date, and it is not desirable to control the dose with the outdated body weight information. Furthermore, there is also a problem where if the patient is using a wheelchair or stretcher, body weight measurement itself is not easy. Therefore, in the present embodiment, in order to address this problem, DL (deep learning) is used to generate a learned model that can deduce the body weight of the patient.

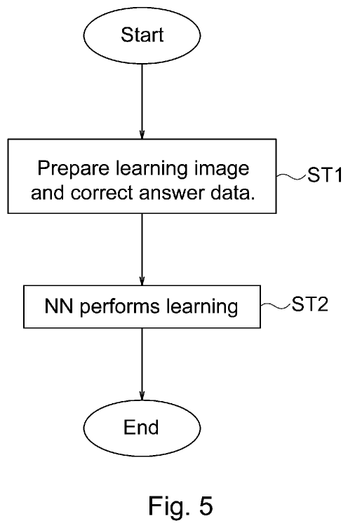

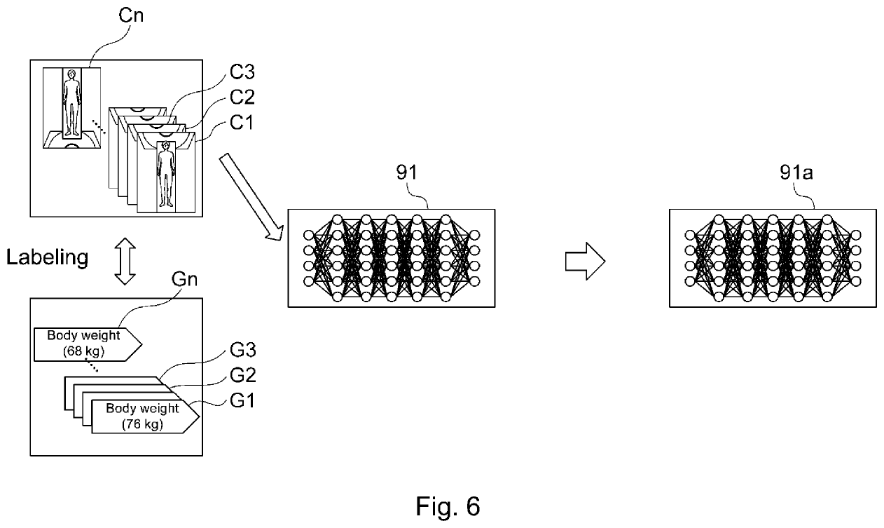

A learning phase for generating a learned model is described below with reference to FIGS. 5 and 6.

FIG. 5 is a diagram showing a flowchart of a learning phase, and FIG. 6 is an explanatory diagram of the learning phase. In step ST1, a plurality of learning images to be used in the learning phase are prepared. FIG. 6 schematically illustrates learning images C1 to Cn. Each learning image Ci (1≤i≤n) can be prepared by acquiring a camera image of a human lying in a supine posture on a table by imaging with a camera from above the table, and executing a prescribed image processing with regard to the camera image. The learning images C1 to Cn include an image of a human in a supine position in a head-first condition and an image of the human in a supine posture in a feet-first condition.

Note that examples of the prescribed image processing to be performed on the camera image include image cropping, standardization processing, normalization processing, and the like. Furthermore, the learning images C1 to Cn include an image of a human in a supine position in a head-first condition and an image of the human in a supine posture in a feet-first condition, as described above. However, a craniocaudal direction of a feet-first human is opposite to the craniocaudal direction of a head-first human. Therefore, in embodiment 1, the prescribed image processing includes a process of rotating an image by 180° in order to match the craniocaudal direction of a human. Referring to FIG. 6, the learning image C1 is head first, while the learning image Cn is feet first. Therefore, the learning image Cn is rotated 180° such that the human craniocaudal direction in the learning image Cn matches the human craniocaudal direction in the learning image C1. Thereby, the learning images C1 to Cn are set up such that the human craniocaudal directions match.

Furthermore, a plurality of correct answer data G1 to Gn are also prepared. Each correct answer data Gi (1≤i≤n) is data representing the body weight of the human in a corresponding learning image Ci of the plurality of learning images C1 to Cn. Each correct answer data Gi is labeled with a corresponding learning image Ci of the plurality of learning images C1 to Cn. After preparing the learning image and correct answer data, the flow proceeds to step ST2.

In step ST2, the computer (learned model generating device) is used to cause a neural network (NN) 91 to execute learning using the learning images C1 to Cn and the correct answer data G1 to Gn, as illustrated in FIG. 6. Thereby, the neural network (NN) 91 executes learning using the learning images C1 to Cn and the correct answer data G1 to Gn. As a result, a learned model 91a can be generated.

The learned model 91a generated thereby is stored in a storage part (for example, a storage part of a CT device or storage part of an external device connected to the CT device).

The learned model 91a obtained from the aforementioned learning phase is used to deduce the body weight of the patient 40 during the examination of the patient 40. An examination flow of patient 40 will be described below.

FIG. 7 is a diagram showing the examination flow. In step ST11, an operator guides the patient 40, who is an imaging subject, into the scan room 100 and has the patient 40 lie on the table 4 in a supine posture as illustrated in FIG. 2.

The camera 6 acquires a camera image of the inside of the scan room and outputs the camera image to the console 8. The console 8 performs prescribed data processing on the camera image received from the camera 6, if necessary, and then outputs the camera image to the display panel 20 of the gantry 2. The display panel 20 can display the camera image in the scan room imaged by the camera 6. After laying the patient 40 on the table 4, the flow proceeds to step ST12.

In step ST12, the body weight of the patient 40 is deduced using the learned model 91a. A method of deducing the body weight of the patient 40 will be specifically described below.

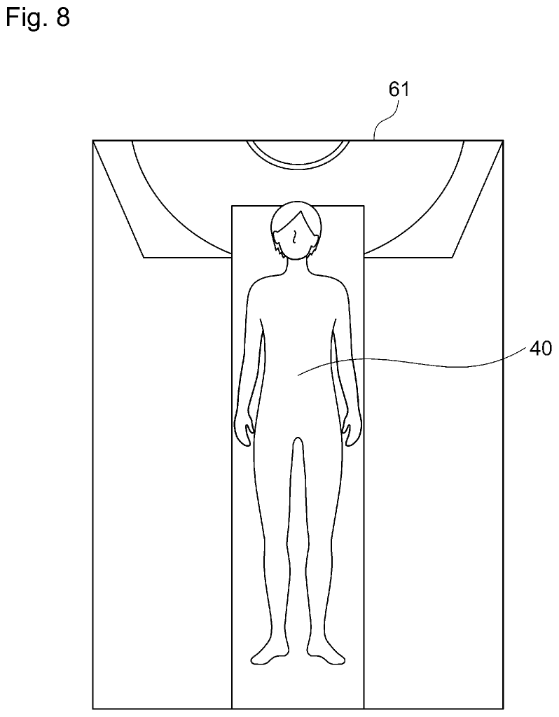

First, as a preprocessing step for deducing, an input image to be input to the learned model 91a is generated. The generating part 841 (refer to FIG. 4) generates an input image used for body weight deducing by executing a prescribed image processing on the camera image obtained by the camera 6. Examples of the prescribed image processing include image cropping, standardization processing, normalization processing, and the like. FIG. 8 is a diagram illustrating a schematic view of a generated input image 61.

Note that when the patient 40 lies on the table 4, the patient 40 gets on the table 4 while adjusting their posture on the table 4, and gets into a supine posture, which is a posture for imaging. Therefore, when generating the input image 61, it is necessary to determine whether or not the posture of the patient 40 in the camera image used to generate the input image 61 is a supine position. Whether or not the posture of the patient 40 is a supine position can be determined using a prescribed image processing technique.

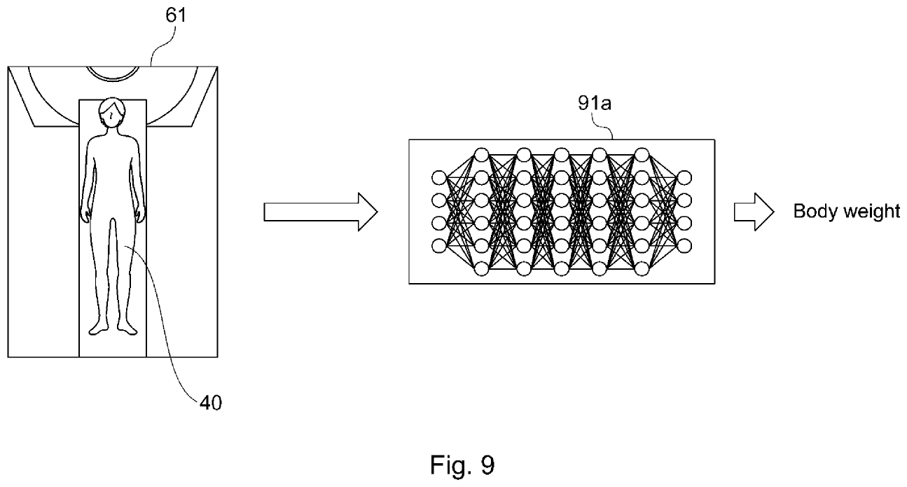

After generating the input image 61, the deducing part 842 (refer to FIG. 4) deduces the body weight of the patient 40 based on the input image 61. FIG. 9 is an explanatory diagram of a deducing phase.

The deducing part 842 inputs the input image 61 to the learned model 91a. Note that in the learning phase (refer to FIG. 6), a foot-first learning image is rotated by 180°. Therefore, if a foot-first input image is generated in the deducing phase, the input image must be rotated by 180°. In the present embodiment, an orientation of the patient 40 is head-first, not feet-first, and therefore, the deducing part 842 determines that rotating the input image by 180° is not necessary. Therefore, the deducing part 842 inputs the input image 61 to the learned model 91a without rotating 180°.

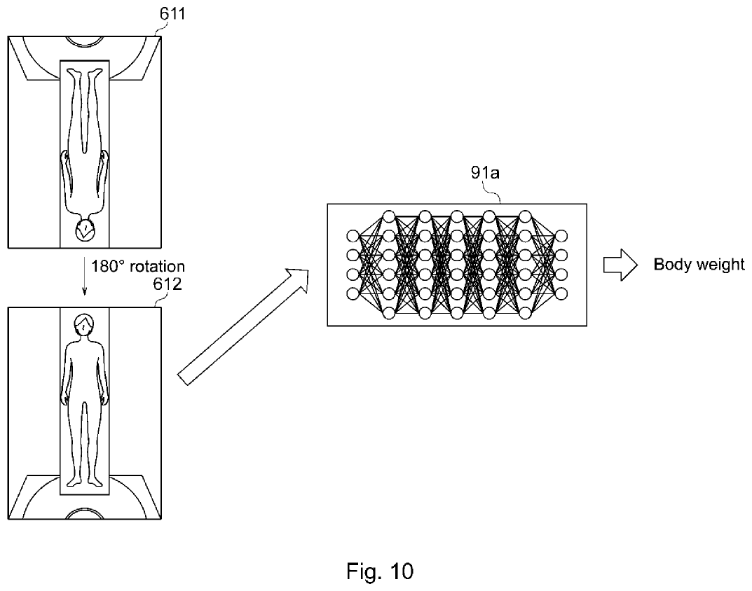

On the other hand, if the orientation of the patient 40 is feet-first, the input image 611 as illustrated in FIG. 10 is obtained. In this case, the input image 612 after rotating the input image 611 by 180° is input to the learned model 91a. Thus, by determining whether to rotate the input image by 180° based on the orientation of the patient 40, the craniocaudal direction of the patient 40 in the deducing phase can be matched to the craniocaudal direction in the learning phase, thereby improving deducing accuracy.

Note that when determining whether to rotate the input image by 180°, it is necessary to identify whether the patient 40 is oriented head first or feet first. The identification method, for example, can be performed based on information in a RIS. The RIS includes the orientation of the patient 40 at the time of the examination, and therefore, the generating part 841 can identify the orientation of the patient from the RIS. Therefore, the generating part 841 can determine whether or not to rotate the input image by 180° based on the orientation of the patient 40.

When the input image 61 is input to the learned model 91a, the learned model 91a deduces and outputs the body weight of the patient 40 in the input image 61. After the body weight is deduced, the flow proceeds to step ST13.

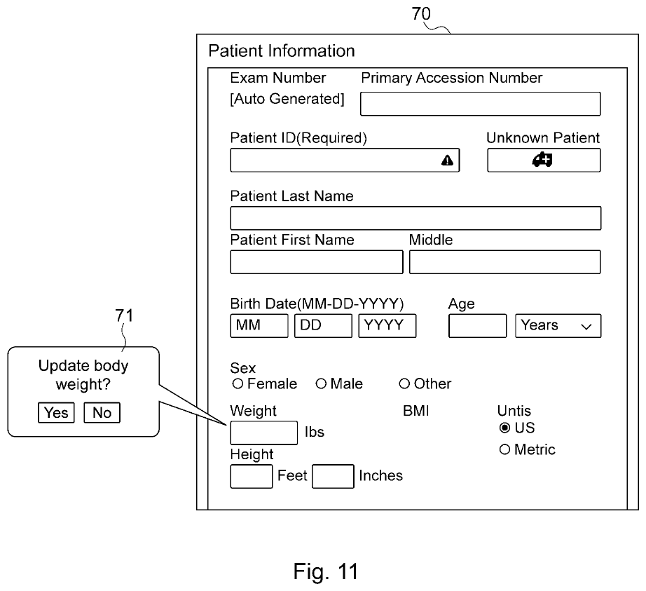

In step ST13, the confirming part 843 (refer to FIG. 4) confirms to the operator whether or not to update the body weight deduced in step ST12. FIG. 11 is an explanatory diagram of a method of confirming to the operator whether or not the body weight is updated.

The confirming part 843 displays patient information 70 on the display part 82 (refer to FIG. 3) in conjunction with displaying a window 71. The window 71 is a window that confirms to the operator whether or not to update the body weight deduced in step ST12. Once the window 71 is displayed, the flow proceeds to step ST14.

In step ST14, the operator decides whether or not to update the body weight. The operator clicks the No button on the window 71 to not update the body weight, and clicks the Yes button on the window 71 to update the body weight. If the No button is clicked, the confirming part 843 determines that the body weight of the patient 40 will not be updated, and the past body weight is saved as-is. On the other hand, if the Yes button is clicked, the confirming part 843 determines that the body weight of the patient 40 is to be updated. If the body weight of the patient 40 is updated, the MS manages the updated body weight as the body weight of the patient 40. Once the body weight update (or cancellation of the update) is complete, the flow proceeds to step ST15.

In step ST15, the patient 40 is moved into the bore 21 and a scout scan is performed. When the scout scan is performed, the reconfiguring part 844 (refer to FIG. 4) reconfigures a scout image based on projection data obtained from the scout scan. The operator sets the scan range based on the scout image. Furthermore, the flow proceeds to step ST16, and a diagnostic scan is performed to acquire various CT images used for diagnosis of the patient 40. The reconfiguring part 844 reconfigures a CT image for diagnosis based on the projection data obtained from a diagnostic scan. Once the diagnostic scan is complete, the flow proceeds to step ST17.

In step ST17, the operator performs an examination end operation. When the examination end operation is performed, various data transmitted to the PACS 11 (refer to FIG. 1) are generated.

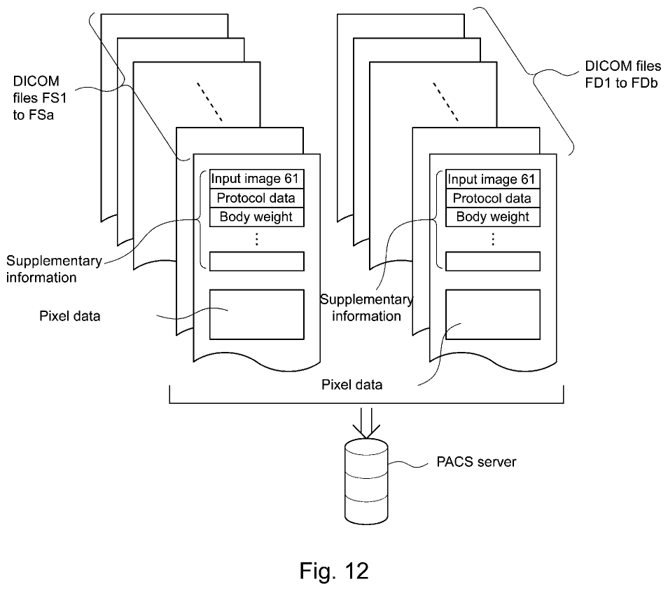

FIG. 12 is an explanatory diagram of an example of various data transmitted to the PACS 11. The X-ray CT device creates DICOM files FS1 to FSa and FD1 to FDb.

The DICOM files FS1 to FSa store scout images acquired in a scout scan, and DICOM files FD1 to FDb store CT images acquired in a diagnostic scan.

The DICOM files FS1 to FSa store pixel data of the scout images and supplementary information. Note that the DICOM files FS1 to FSa store pixel data of scout images of different slices.

Furthermore, the DICOM files FS1 to FSa store patient information described in the examination list, imaging condition information indicating imaging conditions of the scout scan, and the like as data elements of supplementary information. The patient information includes updated body weight and the like. Furthermore, the DICOM files FS1 to FSa also store data elements for supplementary information, such as the input image 61 (refer to FIG. 9), protocol data, and the like.

On the other hand, DICOM files FD1 to FDb store pixel data of the CT images obtained from the diagnostic scan and supplementary information. Note that the DICOM files FD1 to FDb store pixel data of CT images of different slices.

Furthermore, the DICOM files FD1 to FDb store imaging condition information indicating imaging conditions in diagnostic scans, dose information, patient information described in the examination list, and the like as supplementary information. The patient information includes updated body weight and the like. Furthermore, similar to the DICOM files FS1 to FSa, the DICOM files FD1 to FDb also store the input images 61 and protocol data as supplementary information.

The X-ray CT device 1 (refer to FIG. 2) transmits the DICOM files FS1 to FSa and FD1 to FDb of the aforementioned structure to the PACS 11 (refer to FIG. 1).

Furthermore, the operator informs the patient 40 that the examination is complete and removes the patient 40 from the table 4. Thereby, the examination of the patient 40 is completed.

In the present embodiment, the body weight of the patient 40 is deduced by generating the input image 61 based on a camera image of the patient 40 lying on the table 4 and inputting the input image 61 to the learned model 91a. Therefore, body weight information of the patient 40 at the time of examination can be obtained without using a measuring instrument to measure the body weight of the patient 40, such as a weight scale or the like, and thus it is possible to manage the dose information of the patient 40 in correspondence with the body weight of the patient 40 at the time of examination. Furthermore, the body weight of the patient 40 is deduced based on camera images acquired while the patient 40 is lying on the table 4, and therefore, there is no need for hospital staff such as technicians, nurses, and the like to measure the body weight of the patient 40 on a weight scale, which also reduces the workload of the staff.

Embodiment 1 describes an example of the patient 40 undergoing an examination in a supine posture. However, the present invention can also be applied when the patient 40 undergoes examination in a different position from the supine position. For example, if the patient 40 is expected to undergo the examination in a right lateral decubitus posture, the neural network can be trained with learning images for the right lateral decubitus posture to prepare a learned model for the right lateral decubitus position, and the learned model can be used to estimate the body weight of the patient 40 in the right lateral decubitus posture.

In embodiment 1, the operator is asked to confirm whether or not to update the body weight (step ST13). However, the confirmation step may be omitted and the deduced body weight may be automatically updated.

Note that in embodiment 1, the system 10 includes the PACS 11, but another management system for patient data and images may be used instead of the PACS 11.

In embodiment 1, body weight was deduced, but in embodiment 2, height is deduced and body weight is calculated from the deduced height and BMI.

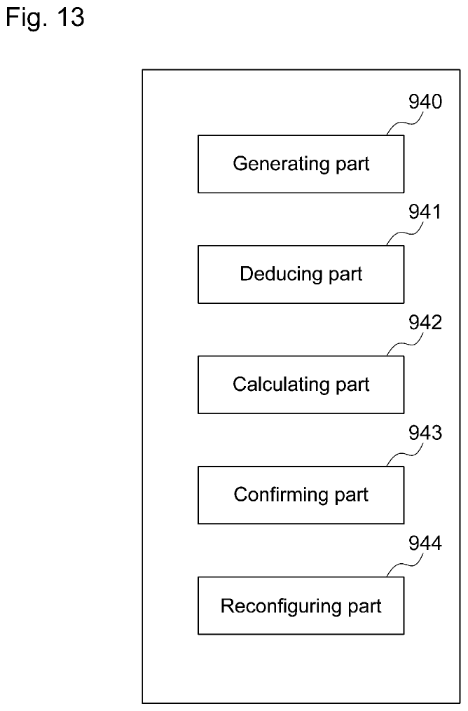

FIG. 13 is a diagram showing main functional blocks of the processing part 84 according to embodiment 2. The processing part 84 has a generating part 940, a deducing part 941, a calculating part 942, a confirming part 943, and a reconfiguring part 944.

The generating part 940 generates an input image to be input to the learned model based on a camera image. The deducing part 941 inputs the input image to the learned model to deduce the height of the patient. The calculating part 942 calculates the body weight of the patient based on the BMI and the deduced height. The confirming part 943 confirms to the operator whether or not to update the calculated body weight. The reconfiguring part 944 reconfigures a CT image based on projection data obtained from a scan.

Furthermore, one or more commands that can be executed by one or more processors are stored in the storage part 83. The one or more commands cause one or more processors to perform the following operations (b1) to (b5): (b1) Generating an input image to be input to the learned model based on a camera image (generating part 940), (b2) Inputting the input image to the learned model to deduce the height of the patient (deducing part 941), (b3) Calculating the body weight of the patient based on the BMI and the deduced height (calculating part 942), (b4) Confirming to the operator whether or not to update the body weight (confirming part 943), (b5) Reconfiguring a CT image based on projection data (reconfiguring part 944).

The processing part 84 of the console 8 can read the program stored in the storage part 83 and execute the aforementioned operations (b1) to (b5).

First, a learning phase according to embodiment 2 will be described. Note that the learning phase in embodiment 2 is also described in the same manner as in embodiment 1, with reference to the flow shown in FIG. 5.

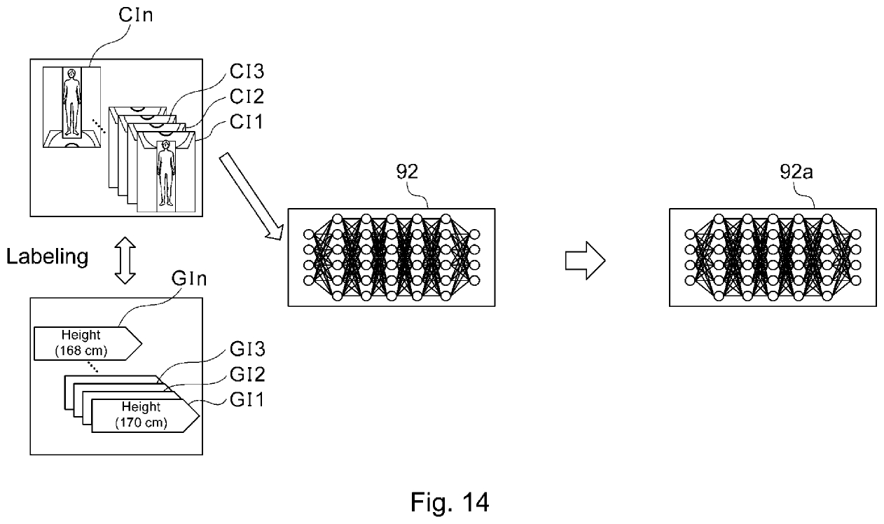

In step ST1, a plurality of learning images to be used in the learning phase are prepared. FIG. 14 schematically illustrates learning images CI1 to CIn. Each learning image CIi (1≤i≤n) can be prepared by acquiring a camera image of a human lying in a supine position on a table by imaging with a camera from above the table, and executing a prescribed image processing with regard to the camera image. In embodiment 2, the learning images C1 to Cn (refer to FIG. 6) used in step ST1 of embodiment 1 can be used as the learning images CI1 to CIn.

Furthermore, a plurality of correct answer data GI1 to GIn are also prepared. Each correct answer data GIi (1≤i≤n) is data representing the height of the human in a corresponding learning image CIi of the plurality of learning images CI1 to CIn. Each correct answer data GIi is labeled with a corresponding learning image CIi of the plurality of learning images CI1 to CIn. After preparing the learning image and correct answer data, the flow proceeds to step ST2.

In step ST2, a learned model is generated. Specifically, as illustrated in FIG. 14, a computer is used to cause a neural network (NN) 92 to execute learning using the learning images CI1 to CIn and the correct answer data GI1 to GIn. Thereby, the neural network (NN) 92 executes learning using the learning images CI1 to CIn and the correct answer data GI1 to GIn. As a result, a learned model 92a can be generated.

The learned model 92a generated thereby is stored in a storage part (for example, a storage part of a CT device or storage part of an external device connected to the CT device).

The learned model 92a obtained from the aforementioned learning phase is used to deduce the height of the patient 40 during the examination of the patient 40. An examination flow of patient 40 will be described below.

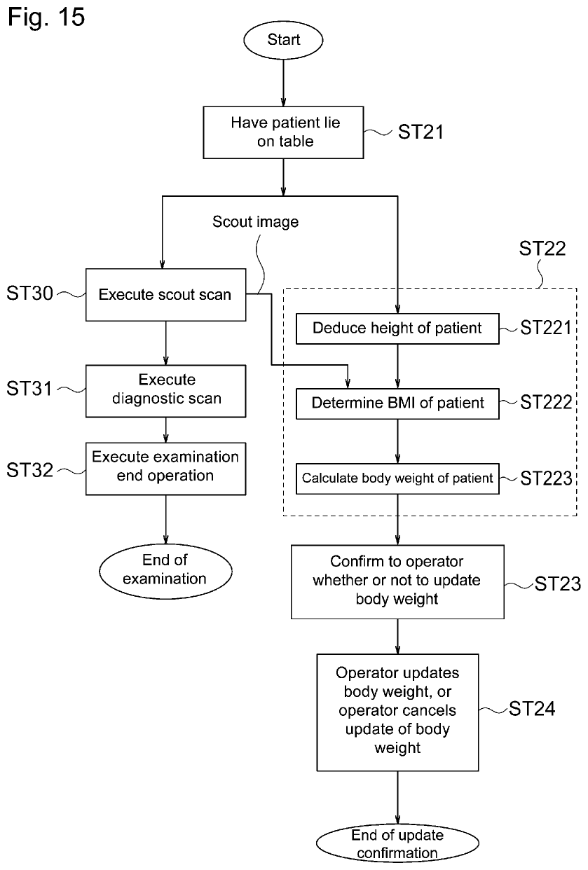

FIG. 15 is a diagram showing an examination flow according to embodiment 2. In step ST21, an operator guides the patient 40 into a scan room and has the patient 40 lie on the table 4. Furthermore, the camera 6 acquires a camera image in the scan room.

After laying the patient 40 on the table 4, the flow proceeds to step ST30 and step ST22.

In step ST30, scanning conditions are set and a scout scan is performed. When the scout scan is performed, the reconfiguring part 944 (refer to FIG. 13) reconfigures a scout image based on projection data obtained from the scout scan. While step ST30 is executed, step ST22 is executed.

In step ST22, the body weight of the patient 40 is determined. A method of determining the body weight of the patient 40 will be described below. Note that step ST22 has steps ST221, ST222, and ST223, and therefore, each step ST221, ST222, and ST223 is described below in order.

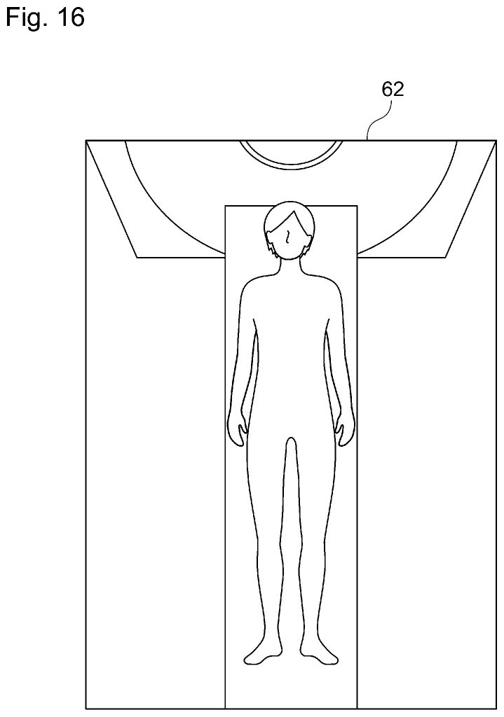

In step ST221, the generating part 940 (refer to FIG. 13) first generates an input image that is input to the learned model in order to deduce the height of the patient 40. In embodiment 2, the posture of the patient 40 is a supine position, similar to embodiment 1. Therefore, the generating part 940 generates the input image used for height deducing by performing a prescribed image processing on the camera image of the patient 40 lying on the table 4 in the supine position. FIG. 16 illustrates a schematic view of a generated input image 62.

Next, the deducing part 941 (refer to FIG. 13) deduces the height of the patient 40 based on an input image 62.

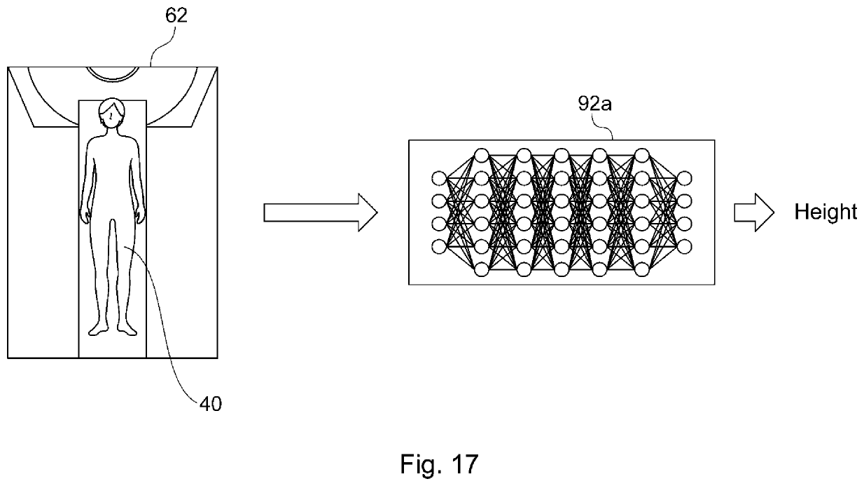

FIG. 17 is an explanatory diagram of a deducing phase of deducing the height of the patient 40. The deducing part 941 inputs the input image 62 to the learned model 92a. The learned model 92a deduces and outputs the height of the patient 40 included in the input image 62. Therefore, the height of the patient 40 can be deduced. Once the height of the patient 40 has been deduced, the flow proceeds to step ST222.

In step ST222, the calculating part 942 (refer to FIG. 13) calculates the Body Mass Index (BMI) of the patient 40. The BMI can be calculated using a known method based on a CT image. An example of a BMI calculation method that can be used includes a method described in Menke J., “Comparison of Different Body Size Parameters for Individual Dose Adaptation in Body CT of Adults.” Radiology 2005; 236:565-571. In embodiment 2, a scout image, which is a CT image, is acquired in step ST30, and therefore, the calculating part 942 can calculate the BMI based on the scout image once the scout image is acquired in step ST30.

Next, in step ST223, the calculating part 942 calculates the body weight of the patient 40 based on the BMI calculated in step ST222 and the height deduced in step ST221. The following relational expression (1) holds between the BMI, height, and body weight.

BMI=body weight÷(height2) (1)

As described above, the BMI and height are known, and therefore, the body weight can be calculated from the expression (1) above. After the body weight is calculated, the flow proceeds to step ST23.

In step ST23, the confirming part 943 confirms to the operator whether or not to update the body weight calculated in step ST22. In embodiment 2, the window 71 (refer to FIG. 11) is displayed on the display part 82, similar to embodiment 1, to allow the operator to confirm the body weight.

In step ST24, the operator decides whether or not to update the body weight. The operator clicks the No button on the window 71 to not update the body weight, and clicks the Yes button on the window 71 to update the body weight. If the No button is clicked, the confirming part 843 determines that the body weight of the patient 40 will not be updated, and the past body weight is saved as-is. On the other hand, if the Yes button is clicked, the confirming part 843 determines that the body weight of the patient 40 is to be updated. If the body weight of the patient 40 is updated, the RIS manages the updated body weight as the body weight of the patient 40.

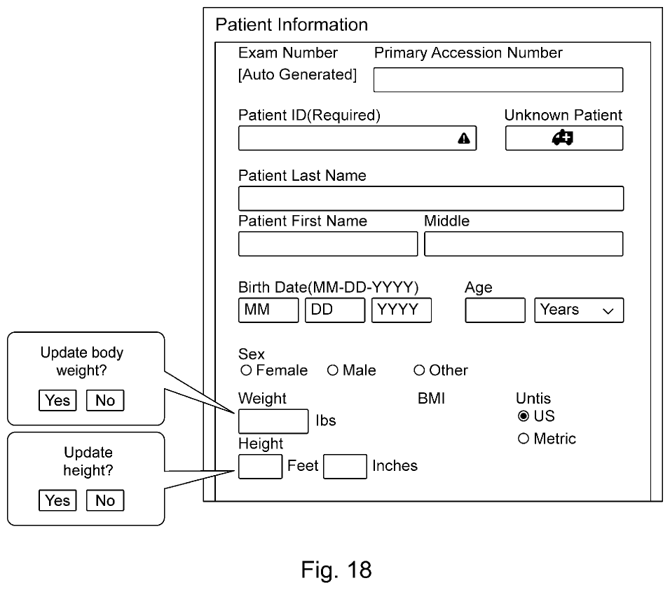

Note that in step ST23, as illustrated in FIG. 18, whether or not the height is updated rather than only the body weight may be confirmed. The operator clicks the Yes button to update the height, or the No button to not update the height. Therefore, patient information for both body weight and height can be managed. Thereby, the flow of the body weight updating process is completed.

Furthermore, while the body weight is being updated, steps ST31 and ST32 are also performed. Steps ST31 and ST32 are the same as steps ST16 and ST17 of embodiment 1, and therefore, a description is omitted. Thereby, the flow shown in FIG. 15 is completed.

In embodiment 2, height is deduced instead of body weight, and body weight is calculated based on the deduced height. Thus, the height may be deduced and the body weight may be calculated from the BMI formula.

Embodiments 1 and 2 assume that the posture of the patient 40 is a supine position. However, depending on the examination to which the patient 40 is subjected, the patient 40 may have to be placed in a different posture than the supine position (for example, the right lateral decubitus position). Therefore, in embodiment 3, a method is described, which can deduce the body weight of the patient 40 with sufficient accuracy, even when the posture of the patient 40 varies based on the examination to which the patient 40 is subjected.

Note that the processing part 84 in embodiment 3 will be described, similarly to embodiment 1, with reference to the functional blocks shown in FIG. 4. In embodiment 3, the following four postures (1) to (4) are considered as postures of a patient during imaging, but another posture may be included in addition to postures (1) to (4): (1) Supine position, (2) Prone position, (3) Left lateral decubitus position, and (4) Right lateral decubitus position.

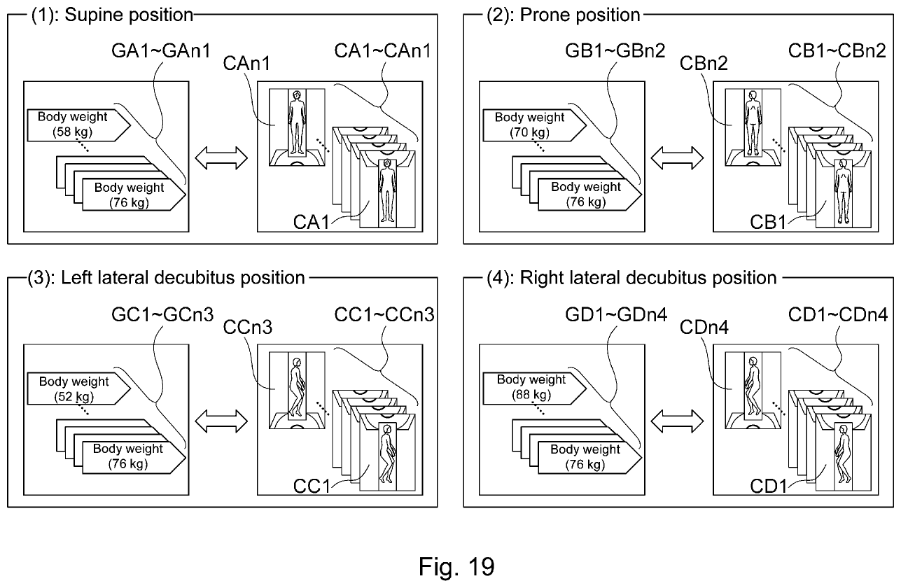

A learning phase according to embodiment 3 will be described below. Note that the learning phase in embodiment 3 is also described in the same manner as in embodiment 1, with reference to the flow shown in FIG. 5. In step ST1, learning images and correct answer data used in the learning phase are prepared. In embodiment 3, for each of the aforementioned postures (1) to (4), a plurality of learning images and correct answer data used in the learning phase are prepared. FIG. 19 is an explanatory diagram of learning images and correct answer data prepared for postures (1) to (4) described above. The learning images and correct answer data prepared for each posture are as follows.

(1) Posture: supine position. n1 number of learning images CA1 to CAn1 are prepared as learning images corresponding to the supine position. Each learning image CAi (1≤i≤n1) can be prepared by acquiring a camera image of a human lying in a supine position on a table by imaging with a camera from above the table, and executing a prescribed image processing with regard to the camera image. The learning images CA1 to CAn1 include an image of a human in a supine position in a head-first condition and an image of the human in a supine posture in a feet-first condition.

Examples of the prescribed image processing to be performed on the camera image include image cropping, standardization processing, normalization processing, and the like. Furthermore, as described above, the learning images CA1 to CAn1 include an image of a human in a supine position in a head-first condition and an image of the human in a supine posture in a feet-first condition. Therefore, the prescribed image processing includes a process of rotating a learning image by 180° in order to match the craniocaudal direction of a human. For example, the learning image CA1 is head first, while the learning image CAn1 is feet first. Therefore, the learning image CAn1 is rotated 180° such that the human craniocaudal direction in the learning image CAn1 matches the human craniocaudal direction in the learning image CA1. Thereby, the learning images CA1 to CAn1 are set up such that the human craniocaudal directions match. Furthermore, correct answer data GA1 to GAn1 are also prepared. Each correct answer data GAi (1≤i≤n1) is data representing the body weight of the human in a corresponding learning image CAi of the plurality of learning images CA1 to CAn1. Each correct answer data GAi is labeled with a corresponding learning image of the plurality of learning images CA1 to CAn1.

(2) Posture: prone position. n2 number of learning images CB1 to CBn2 are prepared as learning images corresponding to a prone position. Each learning image CBi (1≤i≤n2) can be prepared by acquiring a camera image of a human lying in a prone position on a table by imaging with a camera from above the table, and executing a prescribed image processing with regard to the camera image. The learning images CB1 to CBn1 include an image of a human in a prone position in a head-first condition and an image of the human in a prone posture in a feet-first condition.

Examples of the prescribed image processing to be performed on the camera image include image cropping, standardization processing, normalization processing, and the like. Furthermore, as described above, the learning images CB1 to CBn2 include an image of a human in a prone position in a head-first condition and an image of the human in a prone position in a feet-first condition. Therefore, the prescribed image processing includes a process of rotating a learning image by 180° in order to match the craniocaudal direction of a human. For example, the learning image CB1 is head-first, but the learning image CBn2 is feet-first. Therefore, the learning image CBn2 is rotated by 180° such that the craniocaudal direction of the human in the learning image CBn2 matches the craniocaudal direction of the human in the learning image CB1.

Furthermore, correct answer data GB1 to GBn2 are also prepared. Each correct answer data GBi (1≤i≤n2) is data representing the body weight of the human in a corresponding learning image CBi of the plurality of learning images CB1 to CBn2. Each correct answer data GBi is labeled with a corresponding learning image of the plurality of learning images CB1 to CBn2.

(3) Posture: left lateral decubitus position. n3 number of learning images CC1 to CCn3 are prepared as learning images corresponding to a left lateral decubitus position. Each learning image CCi (1≤i≤n3) can be prepared by acquiring a camera image of a human lying in a left lateral decubitus posture on a table by imaging with a camera from above the table, and executing a prescribed image processing with regard to the camera image. The learning images CC1 to CCn3 include an image of a human in a left lateral decubitus posture in a head-first condition and an image of the human in a left lateral decubitus posture in a feet-first condition.

Examples of the prescribed image processing to be performed on the camera image include image cropping, standardization processing, normalization processing, and the like. Furthermore, as described above, the learning images CC1 to CCn3 include an image of a human in a left lateral decubitus posture in a head-first condition and an image of the human in a left lateral decubitus posture in a feet-first condition. Therefore, the prescribed image processing includes a process of rotating a learning image by 180° in order to match the craniocaudal direction of a human. For example, the learning image CC1 is head-first, but the learning image CCn3 is feet-first. Therefore, the learning image CCn3 is rotated by 180° such that the craniocaudal direction of the human in the learning image CCn3 matches the craniocaudal direction of the human in the learning image CC1.

Furthermore, correct answer data GC1 to GCn3 are also prepared. Each correct answer data GCi (1≤i≤n3) is data representing the body weight of the human in a corresponding learning image CCi of the plurality of learning images CC1 to CCn3. Each correct answer data GCi is labeled with a corresponding learning image of the plurality of learning images CC1 to CCn3.

(4) Posture: right lateral decubitus position. n4 number of learning images CC1 to CCn4 are prepared as learning images corresponding to a right lateral decubitus position. Each learning image CDi (1≤i≤n4) can be prepared by acquiring a camera image of a human lying in a right lateral decubitus posture on a table by imaging with a camera from above the table, and executing a prescribed image processing with regard to the camera image. The learning images CC1 to CCn4 include an image of a human in a right lateral decubitus posture in a head-first condition and an image of the human in a right lateral decubitus posture in a feet-first condition.

Examples of the prescribed image processing to be performed on the camera image include image cropping, standardization processing, normalization processing, and the like. Furthermore, as described above, the learning images CD1 to CDn4 include an image of a human in a right lateral decubitus posture in a head-first condition and an image of the human in a right lateral decubitus posture in a feet-first condition. Therefore, the prescribed image processing includes a process of rotating an image by 180° in order to match the craniocaudal direction of a human. For example, the learning image CD1 is head-first, but the learning image CDn4 is feet-first. Therefore, the learning image CDn4 is rotated by 180° such that the craniocaudal direction of the human in the learning image CDn4 matches the craniocaudal direction of the human in the learning image CD1.

Furthermore, correct answer data GD1 to GDn4 are also prepared. Each correct answer data GDi (1≤i≤n4) is data representing the body weight of the human in a corresponding learning image CDi of the plurality of learning images CD1 to CDn4. Each correct answer data GDi is labeled with a corresponding learning image of the plurality of learning images CD1 to CDn4.

Once the aforementioned learning image and correct answer data are prepared, the flow proceeds to step ST2.

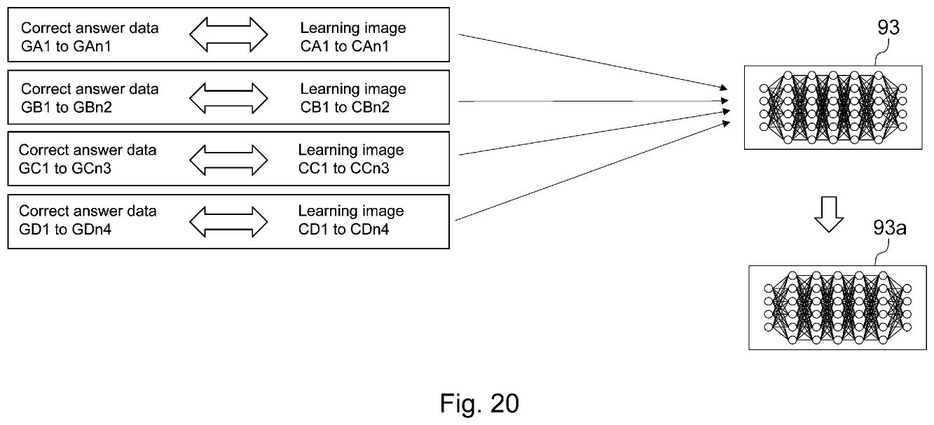

FIG. 20 is an explanatory diagram of step ST2. In step ST2, a computer is used to cause a neural network (NN) 93 to perform learning using learning images and correct answer data (refer to FIG. 19) in the postures (1) to (4) described above. Thereby, the neural network (NN) 93 performs learning using the learning images and correct answer data in the postures (1) to (4) described above. As a result, a learned model 93a can be generated.

The learned model 93a generated thereby is stored in a storage part (for example, a storage part of a CT device or storage part of an external device connected to the CT device).

The learned model 93a obtained from the aforementioned learning phase is used to deduce the body weight of the patient 40 during the examination of the patient 40. An examination flow of the patient 40 will be described below using an example where the posture of the patient is a right lateral decubitus position. Note that the examination flow of the patient 40 in embodiment 3 will also be described with reference to the flow shown in FIG. 7, similar to embodiment 1.

In step ST11, an operator guides the patient 40 into a scan room and has the patient 40 lie on the table 4. A camera image of the patient 40 is displayed on the display panel 20 of the gantry 2. After laying the patient 40 on the table 4, the flow proceeds to step ST12.

In step ST12, the body weight of the patient 40 is deduced using the learned model 93a. A method of deducing the body weight of the patient 40 will be specifically described below.

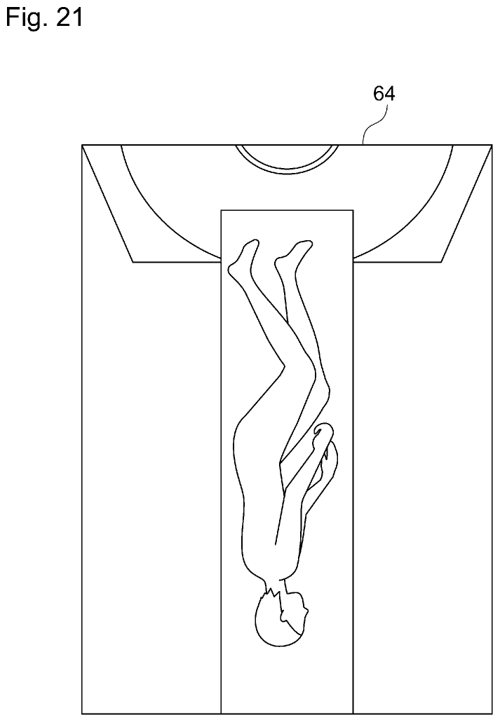

First, an input image to be input to the learned model 93a is generated. The generating part 841 generates an input image used for body weight deducing by executing a prescribed image processing on the camera image obtained by the camera 6. Examples of the prescribed image processing include image cropping, standardization processing, normalization processing, and the like. FIG. 21 illustrates a schematic view of a generated input image 64.

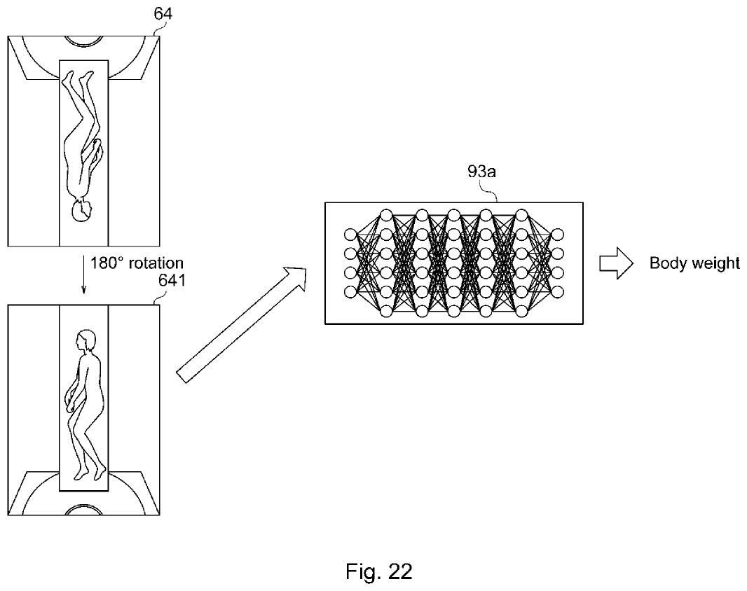

After generating the input image 64, the deducing part 842 (refer to FIG. 4) deduces the body weight of the patient 40 based on the input image 64. FIG. 22 is an explanatory diagram of a deducing phase of deducing the body weight of the patient 40.

The deducing part 842 inputs the input image to the learned model 93a. Note that in the learning phase (refer to FIG. 19), a foot-first learning image is rotated by 180°. Therefore, if a foot-first input image is generated in the deducing phase, the input image must be rotated by 180°. In the present embodiment, the orientation of the patient 40 is feet-first. Therefore, the deducing part 842 rotates the input image 64 by 180° and inputs an input image 641 after rotating by 180° to the learned model 93a. The learned model 93a deduces and outputs the body weight of the patient 40 in the input image 641. After the body weight is deduced, the flow proceeds to step ST13.

In step ST13, the confirming part 843 confirms to the operator whether or not to update the body weight deduced in step ST12 (refer to FIG. 11). In step ST14, the operator determines whether or not to update the body weight. Then, the flow proceeds to step ST15.

In step ST15, the patient 40 is moved into the bore 21 and a scout scan is performed. When the scout scan is performed, the reconfiguring part 844 reconfigures a scout image based on projection data obtained from the scout scan. The operator sets the scan range based on the scout image. Furthermore, the flow proceeds to step ST16, and a diagnostic scan is performed to acquire various CT images used for diagnosis of the patient 40. When the diagnostic scan is completed, the flows proceeds to step ST17 to perform the examination end operation. Thus, the examination of the patient 40 is completed.

In embodiment 3, postures (1) to (4) are considered as patient postures, and learning images and correct answer data corresponding to each posture are prepared to generate the learned model 93a (refer to FIG. 20). Therefore, the body weight of the patient 40 can be deduced even when the posture of the patient 40 is different for each examination.

In embodiment 3, the learned model 93a is generated using the learning images and correct answer data corresponding to the four postures. However, the learned model may be generated using the learning images and correct answer data corresponding to some of the four postures described above (for example, supine position and left lateral decubitus position).

Note that in embodiment 3, body weight is used as the correct answer data to generate a learned model, but instead of body weight, height may be used as the correct answer data to generate a learned model deducing height. Using the learned model, the height of the patient 40 can be deduced even when the posture of the patient 40 is different for each examination, and therefore, the body weight of the patient 40 can be calculated from expression (1) above.

Embodiment 3 indicates an example where the neural network 93 generates a learned model by executing learning using the learning images and correct answer data of postures (1) to (4). In embodiment 4, an example of generating a learned model for each posture is described.

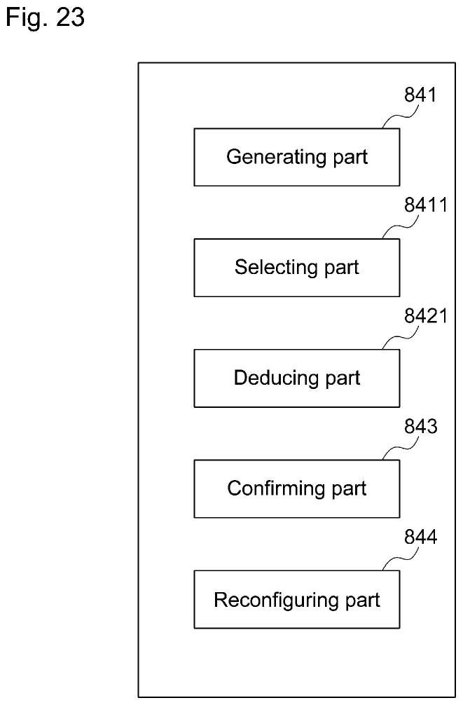

In embodiment 4, the processing part 84 has the following functional blocks. FIG. 23 is a diagram showing main functional blocks of the processing part 84 according to embodiment 4. The processing part 84 of embodiment 4 has the generating part 841, a selecting part 8411, a deducing part 8421, the confirming part 843, and the reconfiguring part 844 as main functional blocks. Of these functional blocks, the generating part 841, the confirming part 843, and the reconfiguring part 844 are the same as embodiment 1, and therefore, a description is omitted. The selecting part 8411 and the deducing part 8421 will be described.

The selecting part 8411 selects, from a plurality of learned models, a learned model to be used for deducing the body weight of the patient. The deducing part 8421 deduces the body weight of the patient by inputting the input image generated by the generating part 841 to the learned model selected by the selecting part 8411.

Furthermore, one or more commands that can be executed by one or more processors are stored in the storage part 83. The one or more commands cause one or more processors to perform the following operations (c1) to (c5): (c1) Generating an input image to be input to the learned model based on a camera image (generating part 841), (c2) Selecting, from a plurality of learned models, a learned model to be used for deducing the body weight of the patient (selecting part 8411), (c3) Inputting the input image to the selected learned model to deduce the body weight of the patient (deducing part 8421), (c4) Confirming to the operator whether or not to update the body weight (confirming part 843), (c5) Reconfiguring a CT image based on projection data (reconfiguring part 844).

The processing part 84 of the console 8 can read the program stored in the storage part 83 and execute the aforementioned operations (c1) to (c5).

A learning phase according to embodiment 4 will be described below. Note that the learning phase in embodiment 4 is also described in the same manner as in embodiment 3, with reference to the flow shown in FIG. 5.

In step ST1, learning images and correct answer data used in the learning phase are prepared. In embodiment 4, postures (1) to (4) illustrated in FIG. 19 are considered as postures of the patient, similar to embodiment 3. Therefore, in embodiment 4, the learning images and correct answer data illustrated in FIG. 19 are also prepared. Once the learning image and correct answer data illustrated in FIG. 19 are prepared, the flow proceeds to step ST2.

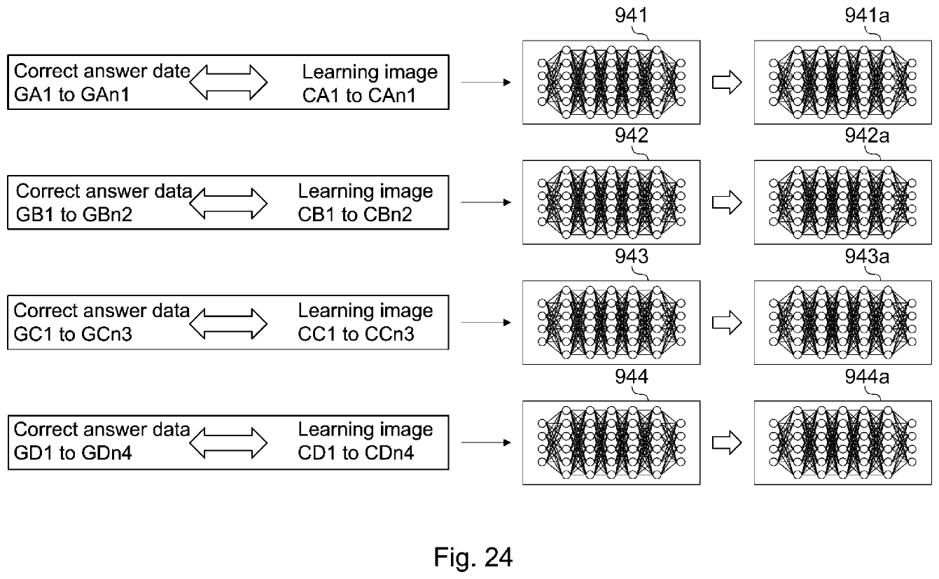

FIG. 24 is an explanatory diagram of step ST2. In step ST2, a computer is used to cause neural networks (NN) 941 to 944 to perform learning using learning images and correct answer data (refer to FIG. 19) in the aforementioned postures (1) to (4), respectively. Thereby, the neural networks (NN) 941 to 944 performs learning using the learning images and correct answer data (refer to FIG. 19) in the postures (1) to (4) described above. As a result, learned models 941a to 944a corresponding to the four postures described above can be generated.

The learned models 941a to 944a generated thereby are stored in a storage part (for example, a storage part of a CT device or storage part of an external device connected to the CT device).

The learned models 941a to 944a obtained from the aforementioned learning phase are used to deduce the body weight of the patient 40 during the examination of the patient 40. An examination flow of patient 40 will be described below.

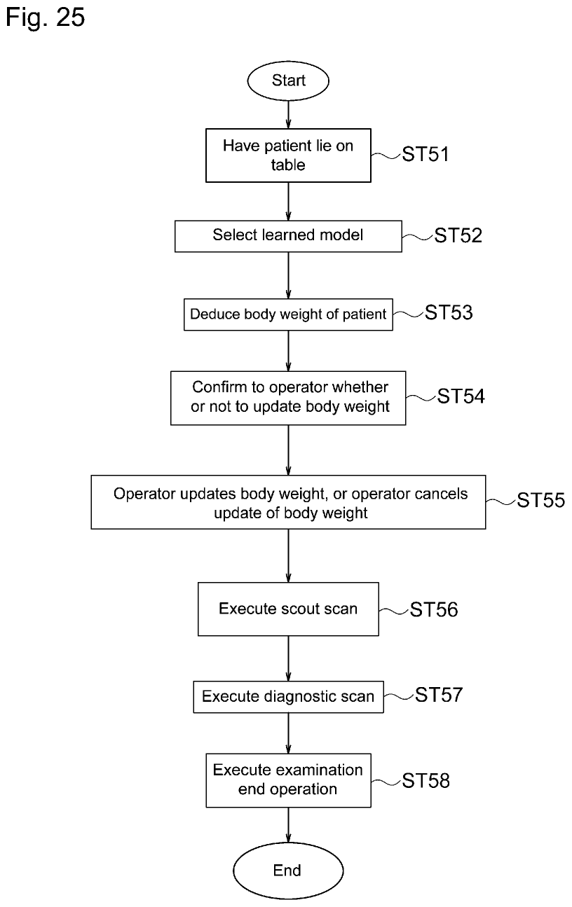

FIG. 25 is a diagram showing an examination flow of the patient 40 according to embodiment 4. In step ST51, an operator guides the patient 40 into a scan room and has the patient 40 lie on the table 4. After laying the patient 40 on the table 4, the flow proceeds to step ST52.

In step ST52, the selecting part 8411 (refer to FIG. 23) selects a learned model used for deducing the body weight of the patient 40 from the learned models 941a to 944a.

Herein, it is assumed that the patient 40 is in the right lateral decubitus position. Therefore, the selecting part 8411 selects the learned model 944a (refer to FIG. 24) corresponding to the right lateral decubitus position from the learned models 941a to 944a.

Note that in order to select the learned model 944a from the learned models 941a to 944a, it is necessary to identify that the posture of the patient is a right lateral decubitus position. The identification method, for example, can be performed based on information in a MS. The MS includes the posture of the patient 40 at the time of the examination, and therefore, the selecting part 8411 can identify the orientation of the patient and posture of the patient from the MS. Therefore, the selecting part 8411 can select the learned model 944a from the learned models 941a to 944a. After selecting the learned model 944a, the flow proceeds to step ST53.

In step ST53, the body weight of the patient 40 is deduced using the learned model. A method of deducing the body weight of the patient 40 will be specifically described below.

First, an input image to be input to the learned model 944a is generated. The generating part 841 generates an input image used for body weight deducing by executing a prescribed image processing on the camera image obtained by the camera 6. In embodiment 4, the posture of the patient 40 is a right prone position, similar to embodiment 3. Therefore, the generating part 841 generates the input image 64 (refer to FIG. 21) to input to the learned model 944a based on a camera image of the patient 40 lying on the table 4 in the right lateral decubitus position.

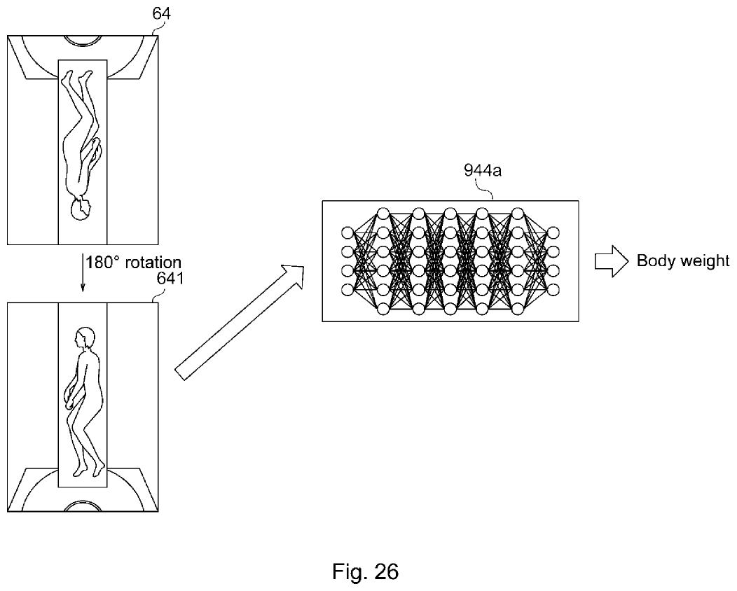

After generating the input image 64, the deducing part 842 (refer to FIG. 23) deduces the body weight of the patient 40 based on the input image 64. FIG. 26 is an explanatory diagram of a deducing phase of deducing body weight.

The deducing part 842 inputs the input image 641 after rotating the input image 64 by 180° to the learned model 944a selected in step ST52 and then deduces the body weight of the patient 40. Once the body weight of the patient 40 has been deduced, the flow proceeds to step ST54. Steps ST54 to ST58 are the same as steps ST13 to ST17 in embodiment 1, and therefore, a description is omitted.

Thus, a learned model may be prepared for each posture of the patient, and the learned model corresponding to the orientation of the patient and posture of the patient during examination may be selected.

Note that in embodiment 4, the body weight is used as the correct answer data to generate a learned model. However, instead of body weight, height may be used as the correct answer data, and a learned model may be generated to deduce the height for each posture. In this case, by selecting the learned model corresponding to the posture of the patient 40, the height of the patient 40 can be deduced even when the posture of the patient 40 is different for each examination, and therefore, the body weight of the patient 40 can be calculated from expression (1) above.

Note that in embodiments 1 to 4, a learned model is generated by a neural network performing learning using a learning image of an entire human body. However, a learned model may be generated by performing learning using a learning image that includes only a portion of the human body, or by performing learning using a learning image that includes only a portion of the human body and a learning image that includes the entire human body.

In embodiments 1 to 4, methods for managing the body weight of the patient 40 imaged by an X-ray CT device were described, but the present invention can also be applied to when the body weight of the patient imaged in a device other than an X-ray CT device (for example, an Mill device) is managed.

In embodiments 1 to 4, deducing is executed by a CT device. However, deducing may be executed on an external computer that the CT device can access through a network.

Note that in embodiments 1 to 4, a learned model was created by DL (deep learning), and this learned model was used to deduce the body weight or height of the patient. However, machine learning other than DL may be used to deduce the body weight or height. Furthermore, a camera image may be analyzed using a statistical method to obtain the body weight or height of the patient.

What is claimed is:

1. A learned model generating method of generating a learned model that outputs a body weight of an imaging subject when an input image of the imaging subject lying on a table of a medical device is input, wherein a neural network generates the learned model by executing learning using:
a plurality of learning images generated based on a plurality of camera images of a human lying on a table of a medical device; and
a plurality of correct answer data corresponding to the plurality of learning images, where each of the plurality of correct answer data represents a body weight of a human included in a corresponding learning image.

2. The learned model generating method according to claim 1, wherein the plurality of learning images includes an image of a human lying on a table in a prescribed posture.

3. The learned model generating method according to claim 2, wherein the plurality of learning images includes an image of the human lying on a table in a different posture from the prescribed posture.

4. The learned model generating method according to claim 3, wherein the plurality of learning images includes at least two of:
a first learning image of the human lying in a supine position;
a second learning image of the human lying in a prone position;
a third learning image of the human lying in a left lateral decubitus position; and
a fourth learning image of the human lying in a right lateral decubitus position.

5. The learned model generating method according to claim 1, wherein the plurality of learning images include an image of the human lying on a table in a head-first condition and an image of the human lying on a table in a feet-first condition.

6. A processing device that executes a process of determining a body weight of an imaging subject based on a camera image of the imaging subject lying on a table of a medical device.

7. The processing device according to claim 6, comprising a learned model that outputs the body weight of the imaging subject when an input image generated based on the camera image is input.

8. The processing device according to claim 7, comprising:
a generating part that generates the input image based on the camera image; and
a deducing part that deduces the body weight of the imaging subject by inputting the input image into the learned model.

9. The processing device according to claim 7, wherein the learned model is generated by a neural network executing learning using:
a plurality of learning images generated based on a plurality of camera images of a human lying on a table of a medical device; and
a plurality of correct answer data corresponding to the plurality of learning images, where each of the plurality of correct answer data represents a body weight of a human included in a corresponding learning image.

10. The processing device according to claim 8, comprising:
a selecting part that selects a learned model used for deducing the body weight of the imaging subject from the plurality of learned models corresponding to a plurality of possible postures of the imaging subject during imaging, wherein
the deducing part deduces the body weight of the imaging subject using the selected learned model.

11. The processing device according to claim 8, comprising a confirming part for confirming to an operator whether or not a deduced body weight is updated.

12. The processing device according to claim 6, comprising:
a deducing part that deduces the height of the imaging subject, containing a learned model that outputs the body height of the imaging subject when an input image generated based on the camera image is input; and
a calculating part that calculates the body weight of the imaging subject based on the height and BMI of the imaging subject.

13. The processing device according to claim 12, wherein the learned model is generated by a neural network executing learning using:
a plurality of learning images generated based on a plurality of camera images of a human lying on a table of a medical device; and
a plurality of correct answer data corresponding to the plurality of learning images, where each of the plurality of correct answer data represents a height of a human included in a corresponding learning image.

14. The processing device according to claim 12, further comprising a generating part that generates the input image based on the camera image.

15. The processing device according to any one of claim 12, comprising:
a reconfiguring part that reconfigures a scout image obtained by scout scanning the imaging subject, wherein
the calculating part calculates the BMI based on the scout image.

16. A storage medium, comprising one or more non-volatile, computer-readable storage media storing one or more commands that can be executed by one or more processors, wherein
the one or more commands cause the one of more processors to execute a process of determining body weight of an imaging subject based on a camera image of the imaging subject lying on a table of a medical device.
